(19) United States
(12) Patent Application Publication
(10) Pub. No.: US 2024/0226843 A1
(43) Pub. Date: Jul. 11, 2024

(54) DIELECTRIC BARRIER DISCHARGE PLASMA REACTOR

(71) Applicant: OFFICE NATIONAL D'ETUDES ET DE RECHERCHES AÉROSPATIALES, PALAISEAU (FR)

(72) Inventors: Paul-Quentin ELIAS, PALAISEAU (FR); Radoslaw DEBEK, PARIS (FR); Maria Elena GALVEZ, PARIS (FR)

(21) Appl. No.: 18/562,814

(22) PCT Filed: Apr. 27, 2022
(86) PCT No.: PCT/FR2022/050802

§ 371 Date: Nov. 20, 2023

(30) Foreign Application Priority Data
May 25, 2021 (FR) 2105438

(51) Int. Cl. B01J 19/08; C01B 3/04; C01B 3/26; C01B 32/05; C01B 32/40; C07C 1/12

(52) U.S. Cl. CPC B01J 19/088; C01B 3/047; C01B 3/26; C01B 32/05; C01B 32/40; C07C 1/12; B01J 2219/0809; B01J 2219/083; B01J 2219/0832; B01J 2219/0875; B01J 2219/0892; B01J 2219/0896; C01B 2203/0277; C01B 2203/0861; C01B 2203/1241

(57) ABSTRACT

A dielectric barrier discharge plasma reactor, for activating a gas-phase chemical reaction, includes at least one tubular pipe made of dielectric material, an inner electrode and an outer electrode. The inner electrode is limited to the inlet of an active zone of the reactor, so that voltage pulses applied between both electrodes generate propagating discharges in the active zone. The reactor produces a volume-contact between a gas stream containing reactants and a plasma created by the discharges, allowing effective transfer of activation energy between the plasma and the reactants.

### TECHNICAL FIELD

This description relates to a dielectric barrier discharge plasma reactor.

### PRIOR ART

Using of a plasma reactor to activate a gas-phase chemical reaction is known. The role of plasma is to provide sufficient activation energy to the reactants so that the reaction occurs more quickly. When the reactor is designed to produce the reaction with a continuous supply of reactants, plasma makes it possible to obtain a higher conversion rate for these reactants, for the same duration of the reactants' presence in the reactor.

Many configurations of plasma reactors have already been proposed. Some issues to consider for these configurations are the following:
obtaining high values for the reactant conversion rate,
obtaining a stable operation of the reactor during continuous operation, and
designing smaller reactors to handle the same amounts of reactants fed to them.
   The proposed configurations vary in particular by the geometry of the electrodes used to generate the plasma, and by the nature of the plasma thus generated. In particular, the following configurations have been proposed for producing plasmas in chemical reactors: dielectric barrier discharge, glow discharge, corona discharge, radiofrequency discharge, microwave discharge, gliding or rotating arc discharge, etc. For example, document EP 1 541 821 A1 describes a reactor with dielectric barrier discharge and a wire-cylinder electrode configuration. In this reactor of EP 1 541 821 A1, the electrode, in the form of a wire, is in contact with the gas stream which contains the reactants, and is parallel to the flow direction of this stream. Positive voltage pulses are applied to the wire electrode relative to an external cylindrical electrode in order to generate the plasma. But because of an accumulation of electrical charges which appears on the surface of the dielectric material in contact with the gas, the plasma is only formed over a limited length between the wire electrode and the dielectric barrier, measured parallel to the wire electrode. For this reason, the duration of the contact between the reactants and the plasma is low, and the conversion rate of the reactants is limited accordingly.

The same limitation occurs for reactor configurations which implement plasmas in the form of layers, and in which the gas stream which contains the reactants crosses the plasma layer substantially perpendicularly to it.

### TECHNICAL PROBLEM

Based on this situation, one object of the present invention is to propose a new type of plasma chemical reactors for which the above disadvantages of prior art reactors are reduced or eliminated.

In particular, the invention aims to provide a plasma chemical reactor having stable operation, and which allows obtaining higher conversion rates for the chemical reactions carried out therein.

### SUMMARY OF THE INVENTION

To achieve this object or other one, a first aspect of the invention proposes a plasma reactor for activating a gas-phase chemical reaction, which comprises:
at least one tubular pipe made of dielectric material, which has a central axis and is arranged to guide a gas stream containing one or more reactant(s) from an inlet end to an outlet end of this tubular pipe, and for each tubular pipe:
/i/ an inner electrode, positioned in the tubular pipe with a radial interval of separation between this inner electrode and the tubular pipe; and
/ii/ an outer electrode, positioned outside the tubular pipe, and arranged to produce an electrical potential which is substantially uniform in a longitudinal segment of an outer surface of the tubular pipe, a volume internal to the tubular pipe which is superposed on this longitudinal segment in an orthogonal projection on the central axis being called the active zone of the reactor.
   This reactor further comprises:
an electrical source, which is connected between the inner electrode and the outer electrode of each tubular pipe.
   The plasma reactor of the invention is therefore of the dielectric barrier discharge type.

According to a first feature of the reactor of the invention, designated by /iii/, the inner electrode is positioned in each tubular pipe near a boundary of the active zone, called the upstream boundary of the active zone, which is oriented towards one among the inlet end and the outlet end. Furthermore, the inner electrode has a spike shape directed towards the other one among the inlet end and the outlet end, and extends parallel to the central axis towards the latter end without exceeding 10% of a length of the active zone, according to a measurement made parallel to the central axis from the upstream boundary of this active zone to the tip of the inner electrode. By means of such a configuration of the electrodes, of the spike-cylinder type, the electrical discharge produced in the gas stream has a propagating discharge configuration, starting from the tip of the inner electrode and extending longitudinally inside the active zone. Such a propagating discharge is composed of an ionization head made up of several filaments, called streamers, and an ionized channel commonly called a leader. The length of the plasma zone can thus be great, enabling the gas stream to be in contact with the plasma inside an entire three-dimensional volume. The activation energy is thus transmitted to the reactants throughout the entire duration of the gas stream's travel through this three-dimensional volume of plasma. Due to the fact that the invention provides such a large volume of plasma, this duration is longer at an equal flow rate of the gas stream, making it possible to obtain higher conversion rates. The limit value of 10% for the inner electrode's protrusion into the active zone ensures that each electrical discharge produced by a voltage pulse applied between the inner electrode and the outer electrode has a longitudinally propagating structure inside the tubular pipe, with no significant radial component at the inner electrode. Preferably, the protrusion of the inner electrode into the active zone, from the upstream boundary of said zone, can be less than 5% of the length of the active zone.

In the context of this invention, the term spike shape for the inner electrode means a protruding shape with a radius of curvature of the convex surface of this inner electrode which is less than 100 μm (micrometers), preferably less than 50 μm and greater than 0.1 μm.

In this description, the inlet and outlet ends of the tubular pipe are so designated in relation to the direction of flow of the gas stream in this tubular pipe. Furthermore, the upstream boundary of the active zone is so designated in relation to the position of the inner electrode, and consequently in relation to the longitudinal extension of the propagating discharge inside the tubular pipe. However, in alternative embodiments of the invention, the upstream boundary of the active zone may be oriented alternately towards the inlet end or towards the outlet end of the tubular pipe, meaning it alternates in being closer to one or the other. In other words, the direction of extension of the propagating discharge from the spike-shaped inner electrode, parallel to the central axis in the tubular pipe, can be in the same direction or in the opposite direction relative to the direction of flow of the gas stream in the tubular pipe.

According to a second feature of the reactor of the invention, designated by /iv/, an inside diameter of the tubular pipe in the active zone of the reactor is between 0.05 mm (millimeters) and 10 mm. This interval constitutes a compromise between the capacity of each tubular pipe for guiding the gas stream with a sufficient flow rate and with limited head loss, and obtaining a plasma which occupies substantially the entire internal cross-sectional area of the tubular pipe.

Finally, according to a third feature of the reactor of the invention, the electrical source is adapted to deliver, during operation of the reactor, voltage pulses which are alternately positive and negative, with a maximum absolute value for the voltage of each pulse which is adapted to produce an electrical discharge in the gas stream, inside the active zone of the reactor, in accordance with a voltage sign convention which corresponds to an electrical potential of the inner electrode from which is subtracted an electrical potential of the outer electrode. Due to the alternation between positive and negative pulses, electrical charges which could accumulate on the inner surface of each tubular pipe made of dielectric material can be neutralized. The plasma reactor can thus have a continuous operation which is stable, with a significant extension of each propagating discharge in the active zone of the reactor, parallel to the central axis. The plasma in each tubular pipe can then occupy a length segment of the active zone which is significant, at the same time as it occupies all or almost all of the cross-sectional area of the tubular pipe in this length segment. In other words, the reactor of the invention allows stable volume-, or three-dimensional, contact between the plasma and the gas stream containing the reactants. Improved conversion rate values can thus be obtained.

Electrical potential which is uniform in a longitudinal segment of the outer surface of the tubular pipe, as produced by the outer electrode, is then understood to mean an electrical potential which presents spatial variations that are less than 10% of the absolute maximum value for the instantaneous voltage of each pulse, inside the longitudinal segment of the outer surface of the tubular pipe. Such an upper limit for the spatial variations of the electrical potential is satisfied in particular before the start of each pulse. This upper limit is compatible with various geometric configurations of the outer electrode, as well as various electrically conductive materials which are possible components of this outer electrode. In particular, it is compatible with an outer electrode made of carbon.

Preferably, the electrical source is adapted so that each positive or negative electrical pulse delivered by the electrical source can be adjusted in order to neutralize electrical charges which would remain on the dielectric material after the previous electrical pulse, or to reverse a sign of the electrical charges that remain on the dielectric material from one pulse to the next, during use of the plasma reactor. Electrical shielding can thus be avoided which could exist on the inner surface of the tubular pipe and limit the plasma volume longitudinally.

Possibly, the reactor may further comprise a catalyst which is placed inside the tubular pipe. The reactor can thus be of the IPC type, for “In-Plasma catalyst”, if the voltage of the positive pulses is sufficient for the propagating discharge to reach the catalyst.

In preferred embodiments of the invention, at least one of the following additional features may optionally be reproduced, alone or with several of them combined:
each electrical pulse delivered by the electrical source during operation of the reactor may have a peak voltage value which is between 1 kV (kilovolts) and 100 kV, preferably between 10 kV and 40 kV, in absolute value;
the electrical source may be adapted to produce the voltage pulses at a frequency which is between 1 Hz (hertz) and 100 kHz, during operation of the reactor;
a length of the inner electrode inside the active zone may be less than 2 mm (millimeters), measured parallel to the central axis between the upstream boundary of this active zone and the tip of the inner electrode;
the inner electrode may consist of a segment of metal wire, for example tungsten or steel wire, having a wire diameter which is between 50 μm (micrometers) and 400 μm;
the length of the active zone may be between 1 mm and 500 mm, preferably between 50 mm and 200 mm, measured parallel to the central axis;
a thickness of the tubular pipe in the active zone may be between 50 μm and 500 μm, measured perpendicularly to the central axis. Such a thickness interval for the dielectric material of the tubular pipe makes it possible to prevent each voltage pulse delivered by the electrical source from having a peak voltage value which is very high so that discharges occur in the gas stream;
the outer electrode may have one of the following forms in the active zone: a wire of electrically conductive material which is wound around the tubular pipe, a sheath of electrically conductive material which surrounds the tubular pipe while being in contact with the outer surface of this tubular pipe, one or more planar metal surface(s) which is (are) in contact with the outer surface of the tubular pipe; and
the dielectric material of the tubular pipe in the active zone may be quartz, glass, or ceramic.

In some embodiments of the invention which accept larger total gas flow rates, the reactor may comprise several tubular pipes arranged in parallel in order to guide respective gas streams simultaneously, each containing the reactant(s). Each tubular pipe is then provided with a respective inner electrode and a respective outer electrode, or with a respective portion of an outer electrode which is shared by several of the tubular pipes, each tubular pipe with the corresponding inner electrode and outer electrode or outer electrode portion satisfying features /i/ to /iv/ mentioned above. In addition, the electrical source is connected between all the inner electrodes on the one hand, and all the outer electrodes or the shared outer electrode on the other hand. The number of tubular pipes in the reactor can thus be between 3 and 400.

A second aspect of the invention proposes a method for implementing a gas-phase chemical reaction, carried out using a reactor which is in accordance with the first aspect of the invention, to activate the chemical reaction. This reaction may in particular be one of the following:
a decomposition of carbon dioxide into carbon monoxide and molecular oxygen;
a reaction between carbon dioxide and hydrogen to produce methane and water;
a reaction to produce molecular hydrogen and solid-state carbon, the gas stream comprising for this purpose at least methane, pure or with at least one additive gas; and
a reaction producing molecular hydrogen, the gas stream comprising for this purpose at least ammonia, pure or with one or more additive gases.

Advantageously, a peak voltage value of each pulse may be adjusted so that, during use of the plasma reactor, this pulse neutralizes electrical charges which would remain on the inner surface of each tubular pipe after the previous pulse, or else reverses the sign of electrical charges remaining on this inner surface of the tubular pipe after the pulse, relative to the previous pulse.

### BRIEF DESCRIPTION OF FIGURES

The features and advantages of this invention will become more clearly apparent from the detailed description below of some non-limiting exemplary embodiments, with reference to the appended figures in which:

FIG. 1 is a longitudinal section view of a basic plasma reactor module according to the invention; and

FIG. 2 is a perspective view which shows several basic modules according to FIG. 1, assembled within a plasma reactor according to the invention.

### DETAILED DESCRIPTION OF THE INVENTION

For clarity sake, the dimensions of the elements represented in these figures correspond neither to actual dimensions nor to actual dimensional ratios. Furthermore, some of these elements are only represented symbolically, and identical references which are indicated in different figures designate identical elements or elements which have identical functions.

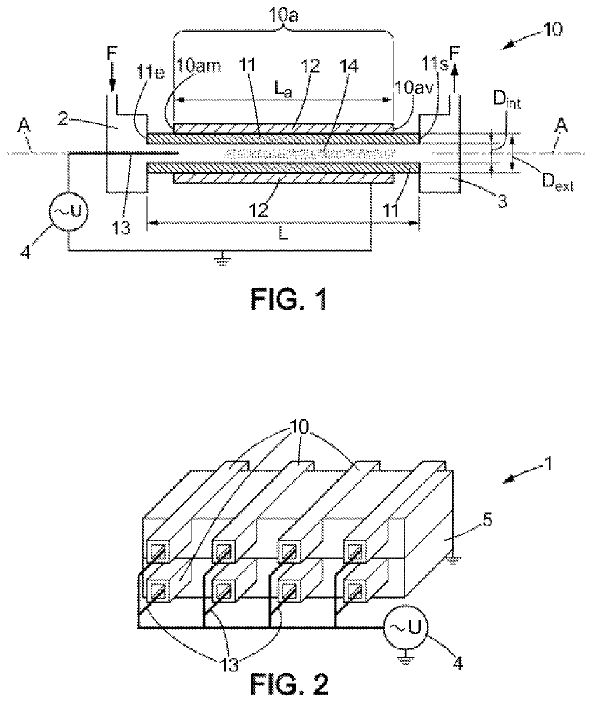

A basic plasma reactor module according to the invention is designated as a whole by the reference 10 in [FIG. 1]. It comprises a tubular pipe 11, for example a tube of glass or alumina (Al₂O₃), which extends between an inlet end 11e and an outlet end 11s. Inlet end 11e leads into a gas stream intake chamber 2, and outlet end 11s leads into a gas stream collection and discharge chamber 3. The connections between inlet end 11e and outlet end 11s to respective chambers 2 and 3 are fluid-tight. A gas stream F containing chemical reactants can thus be introduced continuously into pipe 11. Pipe 11 can be cylindrical with a circular cross-section, having an inside diameter Dint and outside diameter Dext which are respectively equal to 0.6 mm and 1.0 mm, for example, and a tube length which can be equal to 150 mm. However, other cross-section shapes can be used for pipe 11, for example such as a square cross-section (see [FIG. 2]).

An outer electrode 12 is arranged around pipe 11, externally to it, with a geometry adapted to produce an electrical potential which is uniform or substantially uniform over an active zone length Lₐ. For example, outer electrode 12 can be implemented by a metal deposit on the outer surface of pipe 11, so as to form a cylindrical electrode of length Lₐ. An electrical contact can be implemented on outer electrode 12 by any known technology. The segment of pipe 11 which is located within outer electrode 12 has been called active zone of the reactor in the general part of this description, and is designated by the reference 10a. For example, length Lₐ of active zone 10a can be equal to 130 mm, measured between an upstream boundary 10am and a downstream boundary 10av of active zone 10a, the upstream and downstream being oriented relative to the direction of flow of gas stream F in pipe 11. In other words, upstream boundary 10am and downstream boundary 10av of active zone 10a are coincident with the upstream and downstream edges of outer electrode 12.

An inner electrode 13 is arranged in a fixed manner, for example axially, at end 11e of pipe 11. Inner electrode 13 may be superposed on a central axis A-A of pipe 11, and penetrates pipe 11 from inlet end 11e to substantially the level of upstream boundary 10am of active zone 10a. For example, inner electrode 13 exceeds upstream boundary 10am of active zone 10a by 1 mm, in the downstream direction. Inner electrode 13 has a spike shape, its direction also superposable on central axis A-A and oriented downstream. However, an exact superposition of inner electrode 13 with central axis A-A is not essential, and a limited offset between the two does not significantly harm the operation of the plasma reactor. For example, inner electrode 13 can consist of a rigid metal wire, for example tungsten (W) with a diameter of 150 μm. Gas stream F can thus flow between inner electrode 13 and the inner surface of pipe 11.

The rigid metal wire which constitutes inner electrode 13 may have been stretched by locally heating it at the location where it is to be cut to form its spike shape, so that this spike shape has a radius of curvature which is less than 100 μm, for example equal to approximately 20 μm.

An electrical source 4 is connected between electrodes 12 and 13. Preferably, outer electrode 13 is connected to the ground terminal of source 4, so that voltage U delivered by source 4 is equal to the electrical potential of inner electrode 13. Source 4 is selected to deliver voltage pulses which are alternately positive and negative, at a frequency of 50 Hz for example. The peak value of voltage U for each pulse is adjusted to produce a plasma between inner electrode 13 and the inner surface of pipe 11 in active zone 10a. Due to the electrode configuration described above, each pulse produces a propagating electrical discharge in pipe 11, its length dependent on the peak voltage value of the pulses. The length of the propagating discharge can thus be between a few millimeters and almost the entire length Lₐ of active zone 10a. The peak voltage value of each pulse can also be adjusted according to the gas composition of stream F, being between 1 kV and 100 kV, for example equal to 25 kV, in absolute value. In this manner, a volume-contact is produced between a plasma generated by the propagating electrical discharge in pipe 11, and gas stream F. Such a volume-contact allows an efficient transfer of activation energy from the plasma to the reactants contained in gas stream F.

The peak voltage value of each electrical pulse is advantageously adjusted to stabilize a steady state of electrical discharges, in other words to obtain a continuous and stable operation of the plasma reactor. Such stabilization corresponds to the neutralization by each pulse of the electrical charges generated by the previous pulse on the inner surface of pipe 11 in active zone 10a. Preferably, the peak voltage value of each pulse can be adjusted so as to deposit on this inner surface an electrical charge whose sign is opposite to that of the charge left by the previous pulse. Such an inversion, by each pulse, of the electrical charge present on the inner surface of pipe 11 encourages the production of propagating discharges.

[FIG. 2] shows part of a plasma reactor 1 which is composed of the parallel association of several basic modules 10 each in accordance with [FIG. 1]. There can be any number of basic modules 10 in the complete reactor 1, preferably between 3 and 400 inclusive, but [FIG. 2] only shows eight for clarity. Modules 10 may for example be placed in a 2×20 matrix arrangement in a cross-section plane common to all modules 10. Gas stream intake chamber 2 and gas stream collection and discharge chamber 3 may be shared by all modules 10, so that the respective tubular pipes 11 of all modules 10 connect chamber 2 to chamber 3 in parallel, to guide separate gas streams F from one chamber to the other. Furthermore, electrical source 4 can also be shared by all modules 10, with electrical connections which are arranged in parallel to connect the respective electrodes 12 and 13 of all modules 10 to source 4, in connection directions which are identical. According to one possible configuration for reactor 1, each module 10 can be located in a dedicated housing of a supporting structure 5 which is electrically conductive, and which thus ensures electrical contacts with outer electrodes 12 of all modules 10. Supporting structure 5 is advantageously connected to the ground terminal of electrical source 4. In alternative embodiments of reactor 1, supporting structure 5 can directly constitute the outer electrodes 12 of all modules 10.

Plasma reactor 1 can be used for many chemical reactions. The reactants are contained in a gas stream supplied to reactor 1, introduced into intake chamber 2. Such a chemical reaction is commonly called a gas-phase reaction, even if some reaction products may be solid. Some non-limiting examples of gas-phase chemical reactions which can advantageously be carried out in reactor 1 are in particular:

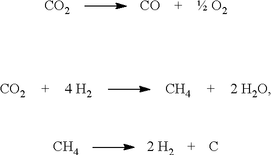

commonly referred to as the Sabatier reaction,

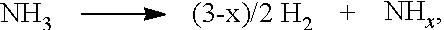

where x is a stoichiometry coefficient between 0 to 3. The gas stream which supplies reactor 1 may consist of the reactants alone, or these reactants diluted in a carrier gas which is inert to the chemical reaction considered. Optionally, other chemical components may be added to the gas stream, to reduce the activation barrier of this reaction or to shift the equilibrium to favor products.

Reactor 1 can be used in combination with a catalyst 14 (see [FIG. 1]), the latter being selected in a known manner according to the chemical reaction considered. For example, but in a non-limiting manner, catalyst 14 may be a powder of nickel (Ni) or an alloy based on at least one noble metal, such as platinum (Pt). Catalyst 14 is then placed inside each pipe 11, preferably in the corresponding active zone 10a, when reactor 1 is oriented so that pipes 11 are substantially horizontal. In this case, reactor 1 is of the IPC type, for “In-Plasma Catalyst”. The catalyst may alternatively be located on the inner surface of each pipe 11, or be located on a divided substrate such as microbeads, a powder with a substrate function, or a foam, for example made of alumina (Al₂O₃) or zirconia (ZrO₂), which is compatible with the flow of each gas stream F.

It is understood that the invention can be reproduced while modifying secondary aspects of the embodiments described in detail above, while retaining at least some of the cited advantages. For example, the described shape of outer electrode 12 of each module 10 and all the cited materials can be adapted or modified, in particular according to the chemical reaction to be implemented in reactor 1. In addition, the detailed description of the invention has only been provided by way of example for embodiments where the direction of flow of the gas stream in each tubular pipe is identical to the direction of extension of the propagating discharge originating from the spike-shaped inner electrode. Indeed, inner electrode 13 was closer to inlet end 11e than to outlet end 11s. However, recall that the direction of extension of the propagating discharge from the spike-shaped inner electrode can also be opposite to the direction of flow of the gas stream in the tubular pipe. In other words, inner electrode 13 can alternatively be located at or near outlet end 11s of tubular pipe 11. Finally, all the numerical values which have been cited have been provided for illustrative purposes only, and can be changed according to the application considered.

1. A dielectric barrier discharge plasma reactor for activating a gas-phase chemical reaction, comprising:
at least one tubular pipe made of dielectric material, which has a central axis and is arranged to guide a gas stream containing one or more reactant(s) from an inlet end to an outlet end of said tubular pipe, and for each tubular pipe (11):
/i/ an inner electrode, positioned in the tubular pipe with a radial interval of separation between said inner electrode and said tubular pipe; and
/ii/ an outer electrode, positioned outside the tubular pipe, and arranged to produce an electrical potential which is uniform in a longitudinal segment of an outer surface of the tubular pipe, a volume internal to the tubular pipe which is superposed on said longitudinal segment in an orthogonal projection on the central axis being called active zone of the reactor,
the reactor further comprising:
an electrical source, which is connected between the inner electrode and the outer electrode of each tubular pipe,
wherein, for each tubular pipe:
/iii/ the inner electrode is positioned in the tubular pipe near a boundary of the active zone, called the upstream boundary of the active zone, which is oriented towards one among the inlet end and the outlet end, said inner electrode having a spike shape directed towards the other one among the inlet end and the outlet end, and extending parallel to the central axis towards said other one among the inlet end and the outlet end without exceeding 10% of a length of the active zone, according to a measurement made parallel to said central axis from the upstream boundary of the active zone to a tip of the inner electrode; and
/iv/ an inside diameter of the tubular pipe in the active zone is between 0.05 mm and 10 mm,
and wherein the electrical source is adapted to deliver, during an operation of the reactor, voltage pulses which are alternately positive and negative, with a maximum absolute value for the voltage of each pulse which is adapted to produce an electrical discharge in the gas stream, inside the active zone, in accordance with a voltage sign convention which corresponds to an electrical potential of the inner electrode from which is subtracted an electrical potential of the outer electrode.

2. The reactor according to claim 1, further comprising a catalyst which is placed inside the tubular pipe.

3. The reactor according to claim 1, wherein the electrical source is adapted to produce the voltage pulses at a frequency which is between 1 Hz and 100 kHz, during the operation of the reactor.

4. The reactor according to claim 1, wherein a length of the inner electrode inside the active zone is less than 2 mm, measured parallel to the central axis between the upstream boundary of said active zone and the tip of the inner electrode.

5. The reactor according to claim 1, wherein the inner electrode consists of a segment of metal wire having a wire diameter which is between 50 μm and 400 μm.

6. The reactor according to claim 1, wherein the length of the active zone is between 1 mm and 500 mm, measured parallel to the central axis.

7. The reactor according to claim 1, wherein a thickness of the tubular pipe in the active zone is between 50 μm and 500 μm, measured perpendicularly to the central axis.

8. The reactor according to claim 1, wherein the outer electrode has one of the following forms in the active zone; a wire of an electrically conductive material which is wound around the tubular pipe, a sheath of an electrically conductive material which surrounds the tubular pipe while being in contact with the outer surface of said tubular pipe, one or more planar metal surface(s) which is (are) in contact with the outer surface of the tubular pipe.

9. The reactor according to claim 1, comprising several tubular pipes arranged in parallel in order to guide respective gas streams simultaneously, each containing the reactant(s), each tubular pipe being provided with a respective inner electrode and a respective outer electrode, or with a respective portion of an outer electrode which is shared by several of the tubular pipes, each tubular pipe with the corresponding inner electrode and outer electrode or outer electrode portion satisfying features /i/to /iv/, and the electrical source being connected between both all the inner electrodes, as well as all the outer electrodes or the shared outer electrode, and wherein the number of tubular pipes in the reactor is between 3 and 400.

10. A method for implementing a gas-phase chemical reaction, carried out using a reactor which is in accordance with claim 1, to activate said chemical reaction.

11. method according to claim 10, wherein the chemical reaction is selected among:
a decomposition of carbon dioxide into carbon monoxide and molecular oxygen;
a reaction between carbon dioxide and hydrogen to produce methane and water;
a reaction to produce molecular hydrogen and solid-state carbon, the gas stream comprising at least methane; and
a reaction producing molecular hydrogen, the gas stream comprising at least ammonia.

12. The reactor according to claim 5, wherein the inner electrode consists of a segment of tungsten or steel wire.

13. The reactor according to claim 6, wherein the length of the active zone is between 50 mm and 200 mm, measured parallel to the central axis.
